(19) United States
(12) Patent Application Publication
(10) Pub. No.: US 2016/0051583 A1
(43) Pub. Date: Feb. 25, 2016

(54) METHOD AND COMPOSITION FOR THE TREATMENT OF SKIN CONDITIONS

(71) Applicant: Kenneth L. Willeford, Little River, SC (US)

(72) Inventor: Kenneth L. Willeford, Little River, SC (US)

(21) Appl. No.: 14/933,063

(22) Filed: Nov. 5, 2015

Related U.S. Application Data
(63) Continuation-in-part of application No. 14/206,290, filed on Mar. 12, 2014.
Application No. 14/206,290 is a continuation-in-part of application No. 12/330,281, filed on Dec. 8, 2008, now abandoned.
Application No. 12/330,281 is a continuation-in-part of application No. 12/166,605, filed on Jul. 2, 2008, now abandoned.

(51) Int. Cl. A61K 33/14; A61K 33/00; A61K 9/08; A61K 33/04

(52) U.S. Cl. CPC A61K 33/14; A61K 33/04; A61K 33/00; A61K 9/08

(57) ABSTRACT

The present invention relates to artificial seawater and its use to treat a variety of skin conditions, when that seawater has more magnesium in it than in naturally occurring seawater. The treatment of acne and the like can be improved with the product of the present invention.

This application is a continuation-in-part of U.S. application Ser. No. 14/206,290 filed on Mar. 12, 2014, which is a continuation-in-part of U.S. application Ser. No. 12/330,281 filed on Dec. 8, 2008, which is a continuation-in-part of U.S. application Ser. No. 12/166,605 filed on Jul. 2, 2008, and they are all incorporated herein in their entirety by reference.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

1. Field of the Invention

The present invention relates to skin care. In particular, the present invention relates to a composition that is useful in treating acne or other facial related infections or conditions.

2. Description of Related Art

Many conditions of the skin are related to alterations in physiology and biochemistry. Trace amounts of minerals, elements, ions, and metals are used in healthy skin metabolism for membrane function, immune modulation and enzyme co-factors. Natural seawater contains a variety of inorganic salts and trace elements in a combined concentration 35 g/L (+−5 g/L) and is hospitable to aquatic life. Application of natural seawater to the skin is acknowledged to be helpful for skin conditions such as acne vulgaris. There does not appear to be information as to why seawater works the way it does, if any particular ingredient is the cause, and if anything other than natural seawater has the same effect. Artificial seawater (also called substitute ocean water ASTM D1141-98 and artificial seawater, as referenced in Tech Talk 27) is a well-known preparation that is an average of the composition of ocean water. By varying the salt concentration g/L +−5% from 35 g/L this method of making artificial seawater generally represents the seawater described above. Artificial seawater's composition excludes sampling from bodies of water called sea or ocean which are not in fact seas or oceans (e.g. the “Dead Sea” which is actually an endorheic lake, and not a sea or ocean, where evaporation and erosion increase the salt concentration above that of an ocean or sea described above, and which is inhospitable to aquatic life) and has a much higher solids concentration than seawater. Artificial seawater is routinely manufactured to approximate the concentrations of many of the components in seawater to produce a product hospitable to life forms (e.g. for use in aquariums), although it does not contain all the ingredients of natural seawater. It generally contains magnesium in a concentration of about 662 g/L +−5%.

Acne vulgaris is a multifactorial inflammatory disease affecting the pilosebaceous follicles. The pilosebaceous is composed of epidermal cells lining the hair follicle and the sebaceous gland. Each hair follicle is associated with one or more sebaceous glands. The infundibulum communicates directly with the epidermis and extends to the opening of the sebaceous gland. In a normal follicle, sebum is secreted from the sebaceous glands and carries desquamated keratinocytes from the follicular epithelium up the follicular canal toward the infundibulum. Keratinocytes are shed as single cells and are moved to the follicle lumen and removed. In acne vulgaris, however, keratinocytes hyperproliferate and are shed as a group of cells rather than individual cells, which obstruct the neck of the follicle. Acne vulgaris develops when the infundibulum becomes occluded, trapping shed cells, sebum, and bacterial products, which leads to inflammation.

The pathogenesis of acne is only partially understood and is multifactorial. When abnormally desquamated keratinocytes accumulate in the sebaceous follicle, comedogenesis occurs and a microcomedo is formed. A microcomedo is a microscopic lesion that is not clinically evident but is the precursor of all acne lesions. Sebum flow is blocked and the follicle becomes plugged with lipids, bacteria, and cell fragments. The microcomedo enlarges and eventually becomes clinically visible. Noninflammatory lesions are an open or closed comedo. Inflammatory acne lesions can be a macule, papule, pustule or nodule.

Several factors have been well established in the pathogenesis of acne. These include sebum production, hormones, bacterial proliferation and inflammation. Sebum is a complex mixture of relatively non polar lipids that is secreted onto the surface of the skin by mature sebocytes. Excess sebum production is involved in the development of acne and is regulated by a number of factors, including local androgens. Both clinical observation and experimental evidence confirm the importance of hormones in the pathophysiology of acne. There is also the proliferation of the bacteria, Propionibacterium acnes, and a Gram-positive anaerobe, which induces inflammation by releasing lipases, proteases, hyaluronidases, and chemotactic factors. Immune cells are activated to induce an inflammatory response.

The proliferation of Propionibacterium acnes within the follicle is responsible for the release of various chemotactic and proinflammatoy mediators. There is a release of chemotactic substances that attracts neutrophils, monocytes, and lymphocytes to the epithelial walls of sebaceous follicles, stimulates the production of proinflammatoy cytokines, stimulates the activation of the complement system, and induces cell mediated immunity. The result of this intensified inflammatory cascade is the disruption of the follicular epithelium, leading to extravasation of lipids, keratinocytes, bacterial antigens, and inflammatory mediators into the surrounding dermis. Follicular rupture and secondary inflammatory responses are responsible for the progression of microcomedones to mature comedones or inflammatory acne lesions.

Many agents are used by physicians for the treatment of acne to focus on pathogenic targets. These agents have been used alone or in combination. Agents used for the hormonal component include estrogens, antiandrogens, and spironolactone. Topical and oral retinoids as well as alpha and beta hydroxyl acids have been used for hyperkeratinization. Sebum production has been treated with topical and oral retinoids, antiandrogens, laser therapy, and photodynamic therapy. Inflammation has been treated with topical and oral antibiotics as well as benzoyl peroxide. Antibacterial agents include topical and oral antibiotics, azelaic acid, benzoyl peroxide, and light therapy.

It is generally acknowledged that minerals support healthy skin metabolism. About 4-5% of the human body is made up of minerals. Many of these act as essential co-factors for enzymatic activity and in normal cellular membrane physiology. Of these minerals, magnesium has been the most widely studied. The precise mechanism and specific minerals involved in maintenance of homeostasis of skin has not been elucidated.

Topical application of natural seawater has also been used for the treatment of acne; however, the clinical effectiveness has not been studied in medical literature. The mechanism of the effect is, therefore, not completely understood. Seawater is a complex mixture of dissolved minerals, metals and ions in a unique composition which varies from location to location. Natural seawater contains about 35 g/L +/−5% of various inorganic salts. Artificial seawater of the same salt concentration, which has not been utilized for the treatment of acne, has its primary use in aquariums for the preservation of aquatic life forms.

Magnesium is the third most abundant mineral in seawater. Typically, magnesium in seawater is present at a level of about 53 mm and frequently is reported at concentrations of 662 ppm +−5%. It is known that higher concentrations of magnesium have a detrimental effect on sea life and, as such, high concentrations of magnesium are not encountered where there is aquatic life and not included in artificial seawater preparations. Studies have shown that elevated concentrations of magnesium have potentially negative biological effects.

Artificial seawater, such as prepared by ASTM D1141-98, has been developed for use in aquariums and where seaweater for other uses is difficult to obtain. Professional marine biologists and reef aquarists are aware that artificial seawater is an imperfect substitute for the perfect medium for marine animal growth, which is pure oceanic water. Attempts to reproduce the chemical composition of natural seawater have provided products that are commercially available. In general, these products make extensive attempts to reproduce the concentration of elements and minerals as they occur under natural oceanic conditions. Because of the potential toxic effects of too much magnesium, magnesium concentrations in artificial seawater are generally at or below concentrations found in naturally occurring seawater, e.g. about 5.2 g/L of MgCl₂ is found in substitute ocean water preparations.

Localized delivery of substances to the hair follicle through the use of liposomes has been developed. Liposomes are microscopic globules of lipids which can be manufactured to enclose substances, such as medications. The lipid nature of liposomes makes them nonpolar. Sebaceous glands are connected to the hair follicle by ducts and release sebum into the upper third portion of the follicular canal, creating an environment rich in neutral nonpolar lipids. Topically applied liposomes are capable of delivering a wide range of drugs, including macromolecules, into hair follicles. Experimental evidence of liposomal delivery into the pilosebaceous unit includes quantitative fluorescence with carboxyfluorescein, which is a negatively charged polar compound. Polar compounds such as seawater have been demonstrated to be delivered to the nonpolar follicular environment of the pilosebaceous unit with the use of liposomes.

### BRIEF SUMMARY OF THE INVENTION

It has been discovered that artificial seawater (substitute ocean water) to which magnesium, such as in the form of MgCl, has been added to a level of at least 8000 parts per million can be used to treat acne. The results with added magnesium are far superior to natural seawater in treating acne and superior to other products containing magnesium. Surprisingly, artificial seawater is at least as effective as natural seawater, even though it has far fewer constituents than natural seawater. Accordingly, both a product and method of treating the skin with artificial seawater and any seawater with a high concentration of magnesium is the main composition and method of treatment of the present invention. Artificial seawater is much easier to prepare and is relatively clean of contaminates unlike seawater.

Accordingly, in one embodiment, the invention is a method for the treatment of acne comprising the application to the affected area of a patient an aqueous composition comprising:

| | |
|---|---|
| Chloride | 19087 ppm +/− 5% |
| Sodium | 11211 ppm +/− 5% |
| Sulfate | 2585 ppm +/− 5% |
| Magnesium | 8000 ppm to 100,000 ppm |
| Calcium | 419 ppm +/− 5% |
| Potassium | 398 ppm +/− 5% |
| Bicarbonate | 146 ppm +/− 5% |
| Bromide | 68 ppm +/− 5%. |

Accordingly, in another embodiment, the invention is an aqueous composition for the treatment of acne comprising:

| | |
|---|---|
| Chloride | 19087 ppm +/− 5% |
| Sodium | 11211 ppm +/− 5% |
| Sulfite | 2585 ppm +/− 5% |
| Magnesium | 8000 ppm to 100,000 ppm |
| Calcium | 419 ppm +/− 5% |
| Potassium | 398 ppm +/− 5% |
| Bicarbonate | 146 ppm +/− 5% |
| Bromide | 68 ppm +/− 5%. |

### BRIEF DESCRIPTION OF THE DRAWINGS

FIG. 1 is a chart showing formulations of artificial seawater and Applicant's invention with additional magnesium.

### DETAILED DESCRIPTION OF THE INVENTION

The present invention relates to the discovery that artificial seawater with added magnesium shows a marked improvement in treating the skin, and especially the treatment of acne, when there is at least about double the amount of magnesium present when compared to artificial seawater mimicking natural seawater (e.g. according to ASTM D1141-98 or any other method known for making artificial seawater). This is true even though artificial seawater does not contain all the ingredients of natural seawater and has not previously been used to treat acne.

While this invention is susceptible of embodiment in many different forms, there is shown in the drawings, and will herein be described in detail, specific embodiments, with the understanding that the present disclosure of such embodiments is to be considered as an example of the principles and not intended to limit the invention to the specific embodiments shown and described. In the description below, like reference numerals are used to describe the same, similar, or corresponding parts in the several views of the drawings. This detailed description defines the meaning of the terms used herein and specifically describes embodiments in order for those skilled in the art to practice the invention.

As used herein the term “about” refers to +−5%.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

References throughout this document to “one embodiment”, “certain embodiments”, and “an embodiment” or similar terms means that a particular feature, structure, or characteristic described in connection with the embodiment is included in at least one embodiment of the present invention. Thus, the appearances of such phrases in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

The drawings featured in the figures are for the purpose of illustrating certain convenient embodiments of the present invention, and are not to be considered as limitations thereto. The term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein and use of the term “means” is not intended to be limiting.

As used herein the phrase “artificial seawater” refers to an aqueous solution which contains a spectrum of minerals, elements, nutrients, micronutrients, and the like in an attempt to, as closely as possible, mimic the composition of naturally occurring seawater. As used herein, ASTM D1141-98 describes a composition of artificial seawater and a method of making it. While artificial seawaters may vary in their exact compositions (i.e. +−5% of each component salt and of the overall salinity), they are designed, in general, to be able to support aquatic marine life that normally lives in an ocean setting. These are readily available from aquatic supply houses and the like and are designed for aquariums and the like, or can be prepared from scratch by means well known in the art, such as the ASTM D1141-98, incorporated herein by reference in its entirety. In one embodiment, the composition of seawater made according to ASTM D1141-98 or the like includes at least the following salts in the following concentrations, plus or minus 5%:
NaCl—24.53 g/L
MgCl₂—5.20 g/L
Na₂SO₄—4.09 g/L
CaCl₂—1.16 g/L
KCl—0.695 g/L
NaHCO₃—0.201 g/L
KB_{R}—0.101 g/L
H₃BO₃—0.027 g/L
SRCl₂—0.025 g/L
NaF—0.003 g/L
   The final salinity of which is about 35 g/L +/−5%. Optionally, heavy metals can be added in accordance with ASTM D1141-98, but not limited to about the following concentration:
Ba(NO₃)₂—0.0000994 g/L
Mm(NO₂)₂—0.0000340 g/L
Ca(NO₃)₂—0.0000308 g/L
Zn(NO₃)₂—0.0000096 g/L
Pb(NO₃)—0.0000066 g/L
Ag (NO₃)—0.00000049 g/L

Specifically, the present invention will be in an aqueous composition comprising a method for the treatment of acne comprising the application to the affected area of a patient an aqueous composition comprising:

| | |
|---|---|
| Chloride | 19087 ppm +/− 5% |
| Sodium | 11211 ppm +/− 5% |
| Sulfate | 2585 ppm +/− 5% |
| Magnesium | 8000 ppm to 100,000 ppm |
| Calcium | 419 ppm +/− 5% |
| Potassium | 398 ppm +/− 5% |
| Bicarbonate | 146 ppm +/− 5% |
| Bromide | 68 ppm +/− 5%. |

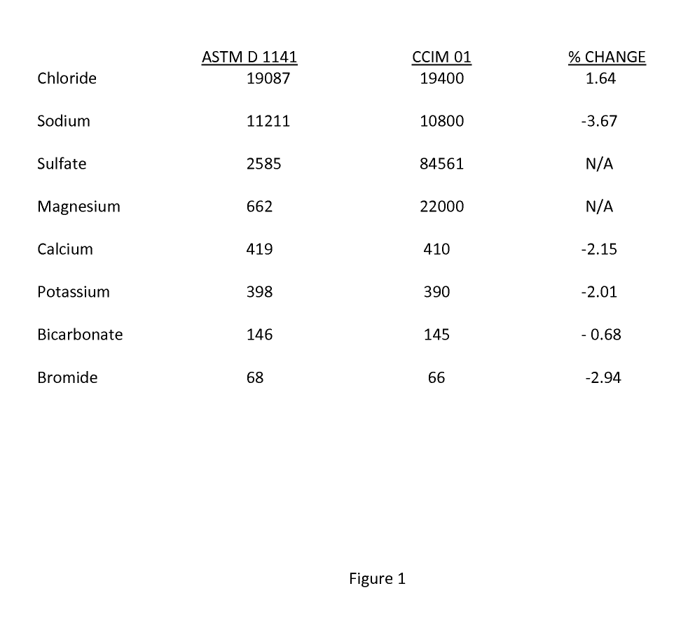

As used herein “magnesium” refers to the magnesium ion and, in most cases, the Mg⁺⁺ion that is introduced into the artificial or natural seawater in the form of a magnesium salt. Examples of magnesium salts useful in the present invention are magnesium oxide, magnesium carbonate, magnesium chloride, magnesium sulfate, (such as magnesium sulfate heptahydrate, and anhydrous magnesium sulfate) magnesite and dolomite. While seawater is normally no more than about 1,300 parts magnesium per million, magnesium is added to the solution in the present invention in an amount to raise a sample of seawater to at least about 8,000 parts magnesium per million. In some embodiments, the magnesium could be at least about 8,000 parts per million, 20,000, 40,000, 50,000, 80,000, 100,000 or more. In the embodiment in the example, and in FIG. 1, magnesium is present as a salt at a rate of 22,000 ppm. In other words, the present invention includes embodiments wherein the concentration of magnesium is at least about 6-8 times the normal naturally occurring magnesium in seawater, but the remaining elements of seawater remain unchanged.

Magnesium (such as magnesium chloride or magnesium sulfate) is added to the artificial seawater in such a manner that it dissolves the appropriate amount of magnesium at 8,000 parts per million and above. Depending on the magnesium salt selected, this could be done at room temperature with stirring, or could be done with heat or other means to achieve the desired concentration of magnesium. The optimum amount of magnesium will depend on the particular skin condition being treated, the salt selected, the sensitivity of the patient, the frequency of treatment and the like, however, one skilled in the art with this disclosure could easily optimize the amount of magnesium in view of the present disclosure. Thus the amount of magnesium and the salt ion (such as sulfate or chloride) is elevated above the HSTM level. The ion salt elevation is based on the amount of magnesium salt added.

Applying the composition of the present invention can be in any convenient topical method. So, for example, for treatment of facial acne the solution can be applied to the face once a day twice a day, or as often as desired, to achieve the desired effect. It can be applied and left on, or applied and rinsed off, or can be used as part of a facial wash in combination with soaps or other skin treatments. Application can be with the hands, or by means of the solution applied to cotton balls, cotton swabs, rinsing, or vaporization. In addition, the solution of the present invention could be warmed or otherwise heated so that the pores of the skin will open up during treatment with the present invention.

As used herein, “treating the skin” refers to treatment of skin conditions which show improvement with application of the product of the present invention. The present invention is primarily a treatment for acne, but treatment as a preventative for skin conditions is also within the scope of the invention. Conditions of excess oil, other skin infections, eczema, rosacea, psoriasis, seborhhea and the like can also be treated on the skin with the present invention.

The composition of the present invention can also be formulated into a liposome type formulation. As used in the present invention, the term “liposome” means a vesicle composed of amphiphilic lipids arranged in a spherical bilayer or bilayers.

Liposomes are unilamellar or multilamellar vesicles which have a membrane formed from a lipophilic material and an aqueous interior. The aqueous portion contains the present composition to be delivered. Cationic liposomes possess the advantage of being able to fuse to the cell wall. Non-cationic liposomes, although not able to fuse as efficiently with the cell wall, are taken up by macrophages in vivo. Selection of the appropriate liposome depending on the agent to be encapsulated would be evident given what is known in the art.

Liposomes are useful for the transfer and delivery of active ingredients to the site of action. Because the liposomal membrane is structurally similar to biological membranes, when liposomes are applied to a tissue, the liposomes start to merge with the cellular membranes. As the merging of the liposome and cell progresses, the liposomal contents are emptied into the cell where the active agent may act.

Another embodiment also contemplates the use of liposomes for topical administration. Such advantages include reduced side-effects related to high systemic absorption of the administered drug, increased accumulation of the administered drug at the desired target, and the ability to administer a wide variety of drugs, both hydrophilic and hydrophobic, into the skin. Several reports have detailed the ability of liposomes to deliver agents, including high-molecular weight DNA into the skin. Compounds including analgesics, antibodies, hormones and high-molecular weight DNAs have been administered to the skin. The majority of applications resulted in the targeting of the upper epidermis.

### EXAMPLES

Aqueous formulations, as described in FIG. 1 and in paragraph [025], can be made by techniques well known in the art (such as ASTM D1141-98 with and without having metal trace minerals). Magnesium salt is added to the compositions with magnesium in the form of the magnesium chloride salt and/or the sulfate salt sufficient to reach the indicated concentrations of magnesium ion. Individuals are treated with one of the four formulations for conditions of acne and the result after daily treatments over four weeks are compared. Compositions comprising natural and artificial seawater are comparable with artificial seawater performing slightly better than natural seawater. Individuals treated with either artificial or natural seawater with additional magnesium show marked improvement compared with the compositions without the addition of magnesium.

The present invention compositions have also been determined to be useful for the treatment of skin wrinkles. Skin wrinkles are one aspect of skin aging. Skin aging is known to be from both intrinsic factors and photoaging factors. Intrinsic factors include all clinical, histologic and physiologic changes in the sun-protected skin of adults. These include a decline in the ability to repair damaged DNA and abnormalities in transepidermal water loss. It is the water loss and effects of dehydration that contribute to the appearance of skin wrinkles. Photoaging has a major impact on appearance causing wrinkles, hyperpigmentation and a leathery look. There are exaggerated functional losses, as seen in intrinsic aging, with further loss of immune function, and inflammation as a result of ultraviolet radiation.

Synthetic seawater, as described herein, appears to be useful in the treatment of aging skin through many mechanisms. While not wanting to be held to a particular theory, the following seems to explain the results obtained with treatment with synthetic seawater. These include anti-inflammatory support, anti-oxidant activity, enhanced molecular and cellular detoxification and scavenging, immunostimulation, as well as osmotic effects of cellular water.

The beneficial anti-inflammatory and antioxidant effects of synthetic seawater appear to be mediated though the apparent supplementation of trace minerals. Trace mineral deficiency is common and is from a combination of poor nutrition and depleted soils. Trace mineral deficiency is well known to have adverse effects on the human anti-inflammatory and anti-oxidant biochemical pathways. These have been extensively studied with zinc and selenium as common examples. The importance of selenium as an essential trace element has been published in biomolecular chemistry journals reporting the mechanism through which selenium exerts its redox activities. There are several selenocysteine-containing enzymes such as glutathione peroxidase, iodothyronine deiodinase and thioredoxin reductase. All these enzymes have selenocysteine in the active sites.

Selenium's role as an essential nutrient is as a result of its unique chemistry enabled by the presence of selenium in selenoproteins. Epidemiological findings have linked inadequate status of selenium to increased risk of cancer. The protective action of selenium is a combination of various mechanisms. Amongst all the diverse mechanisms that have been proposed, some important ones are (a) the protective role of selenoproteins/selenoenzymes, (b) induction of apoptosis, (c) immune system effects, (d) detoxification of antagonistic metals, (e) inactivation of nuclear transcription factor, (f) regulation of lipoxygenases, (g) effect on advanced cancer condition, (h) reduction of oxidative stress, (i) induction of Phase II enzymes, (j) androgen receptor down regulation, (k) inhibition of DNA adduct formation, and (I) cell cycle arrest. Many of these effects are directly applicable to the benefits concerning the cellular molecular biology of skin.

Other enzymes with anti-inflammatory and antioxidant effects which use zinc and selenium as co-factors include superoxide dismutase, glutathione peroxidase and malondialdehyde. This is not a comprehensive list of enzymes involved in the anti-inflammatory and antioxidant biochemical pathways, but these are used as examples. Many more enzymes are involved and each has trace minerals as co-factors. Selenium and zinc are not the only two co-factors, and synthetic seawater has many trace elements and minerals. The important concept is that inflammation and oxidative damage are components of a complex process, with skin aging which results in skin wrinkles. Synthetic seawater has been determined to replenish many trace minerals and elements which are essential co-factors for enzymatic function which helps explain the novel benefits observed with artificial seawater treatments of skin.

Nitric oxide is another enzymatically produced substance with beneficial effects on skin aging. It is another antioxidant, but has many other properties as well. The effect of nitric oxide on epithelial cells has been studied. In burn wounds there is enhanced re-epithelialization though follicle stem cell recruitment, increased number of procollogen-expressing fibroblasts, increased angiogenesis, enhanced hair follicle regeneration, and promotion of wound bed infiltration and retention with growth factors and cytokines during the healing process. Nitric oxide is produced enzymatically by nitric oxide synthetase and requires co-factors as with other enzymes. There is experimental evidence in human skin fibroblasts that nitric oxide protects against the injurious effects of ultraviolet A radiation. This nitric oxide dependent mechanism was confirmed by the observation in nitric oxide depleted cultures of increased susceptibility to ultraviolet A induced lipid peroxidation. The molecular mechanisms of endothelial dysfunction from nitric oxide synthetase inhibition have also been published in medical literature. These effects of nitric oxide are a result of nitric oxide production through enzymatic function of nitric oxide synthetase, are dependent upon trace elements for co-factors, and appear to be supported by topical administration of synthetic seawater.

Molecular detoxification is also involved in skin aging. There is an age-dependent decline in the activity of the hydrogen peroxide detoxifying antioxidant catalase in chronically sun-exposed epithelium. Catalase mimetics, as well as peroxynitrite scavengers, are thought to maintain hydrogen peroxide detoxification pathways. Creatinine kinase activity is also lost through oxidative stress. These examples of additional enzymes involved in the detoxification process support the concept of micronutrient and trace mineral supplementation for optimal enzymatic activity to prevent loss of skin homeostasis and could be another explanation of the mechanism that artificial seawater treats skin wrinkles.

Another mechanism of protection against epidermal ultraviolet B photodamage, as well as water loss, is mediated though caspase-14. Caspase-14 is an aspartate-specific proteinase whose expression is restricted almost exclusively to the suprabasal layers of the epidermis and the hair follicles. The proteolytic activation of caspase-14 is associated with stratum corneum formation indicating caspase-14 is essential for keratinocyte differentiation and cornification. Caspase-14 is dependent upon a proteinase enzyme for formation and has beneficial effects in protection against ultraviolet B radiation by controlling the ultraviolet B scavenging capacity of the stratum corneum.

Caspase-14 has an additional mechanism which is beneficial in the treatment of wrinkles by increasing cell water. This effect is seen experimentally as caspase-14 deficient epidermis is characterized by reduced skin-hydration levels and increased water loss. Cellular biology has revealed the mechanism as being mediated by the ability of caspase-14 to cleave profilaggrin. This mechanism is supported by the observation that altered profilaggrin processing patterns have been linked to caspase-14 deficient epidermis. The importance of the filaggrin structure in moisturization of the skin has been established and is another example of an enzymatically dependent pathway involved in the prevention of skin aging. Once again artificial seawater may be supplying the necessary elements to achieve reduction of wrinkles by this method.

The severity of skin wrinkles is usually considered related to cellular water. Skin is the limiting tissue of the body and within skin, the stratum corneum (SC) of the epidermis is the limiting barrier to water loss. Water homeostasis of the epidermis is important for the appearance and physical properties of the skin, as well as for water balance in the body. The transport of water is dependent upon gap junctions and cellular membrane channels. Aquaporin-3 (AQP3) is a membrane transporter of water expressed in plasma membranes in the basal layer keratinocytes of epidermis in normal skin. Gap junctions and plasma membrane channels are composed of proteins. The tertiary structure of proteins is dependent upon the chemical environment and the permeability is sensitive to electrolyte ion concentrations. For example, the permeability of gap junctions is regulated by calcium ion. Likewise, calcium membrane pumps are regulated by magnesium ion concentrations. Intracellular water transport and homeostasis is maintained by membrane transporters and channels and these are made of proteins whose structure and function are dependent upon the chemical environment composed of elements and minerals.

There is also an osmotic component to skin wrinkles where severity of wrinkles is decreased with increased cellular water and the amount of cellular water can be increased along osmotic concentration gradients. Topical application of synthetic seawater would be expected to increase cellular water through this mechanism, in addition to the biochemical and cellular molecular mechanisms previously discussed. Transport across liquid membranes is described by Fick's Law, the Nerst-Planck equation, or the Einstein equation to predict diffusivity depending upon electrochemical potentials and concentrations. Essentially, the permeability is a function of the solute concentration, the area of contact and the contact time.

These mechanisms of anti-inflammatory support, anti-oxidant activity, enhanced molecular and cellular detoxification and scavenging, immunostimulation, as well as osmotic effects of cellular water, are all supported with the topical administration of synthetic seawater. This is the cellular molecular basis of the benefits of synthetic seawater with added magnesium for applications associated with skin homeostasis. One such application would be for the treatment of skin wrinkles.

It has also been discovered that the present invention relates to the use of the present invention for use as a sunscreen. The mechanism for this use is postulated to be similar as for skin wrinkles with special emphasis on the antioxidant properties. Antioxidants are known to decrease the severity of ultraviolet damage and to promote healing of epithelial tissues.

The present invention, artificial seawater, is also useful for the treatment of hair loss. Hair follicle stem cell recruitment and enhanced hair follicle regeneration is seen with nitric oxide as has been discussed. In addition, Caspase-14 is an aspartate-specific proteinase whose expression is restricted almost exclusively to the suprabasal layers of the epidermis and the hair follicles. Each of the pilosebaceous units discussed concerning acne vulgaris also contains a hair follicle and improvements in pilosebaceous unit function would also be expected to improve hair growth.

In addition to these cosmetic uses there are many medical uses of synthetic seawater with added magnesium. Normal saline is commonly used for medical applications. Synthetic seawater would be superior in these applications as the benefits of the sodium and chloride are maintained and there are additional benefits from the trace minerals and elements. The benefits are similar to those described concerning the cellular molecular biology of skin.

The present invention would be useful for peritoneal lavage, sinus surgery, cystoscopy, ureteroscopy, colonoscopy, bronchoscopy, laryngoscopy, hysteroscopy, arthroscopy, an eye lubricant or irrigant, a source of water and electrolytes, priming for dialysis, and IV fluids. Normal saline is used for many of these indications because of improved visualization. Synthetic seawater would have an added advantage by promoting healing of the tissues being treated and the mechanism of improved tissue healing is multifactorial as discussed.

These same mechanisms of anti-inflammatory support, anti-oxidant activity, enhanced molecular and cellular detoxification and scavenging, immunostimulation, as well as osmotic effects of cellular water, which are all supported with the topical administration of synthetic seawater with added magnesium, would support the use of synthetic seawater with added magnesium in medical applications where normal saline is currently used.

The concentration could be adjusted for individual uses. For example, a concentrated solution would be more beneficial for use for skin wrinkles. A diluted solution would have applicability for eye irrigation. A general formula, nX could be used where X is the standard concentration as listed in FIG. 1 and n is a fraction when diluted solution is need for sensitive tissues such as the eye or for intravenous use. A value of n greater than one would be concentrated solutions such as for use with skin wrinkles.

What is claimed is:

1. A method for the treatment of acne comprising the application to the affected area of a patient an aqueous composition comprising:

| | |
|---|---|
| Chloride | 19087 ppm +/− 5% |
| Sodium | 11211 ppm +/− 5% |
| Sulfate | 2585 ppm +/− 5% |
| Magnesium | 8000 ppm to 100,000 ppm |
| Calcium | 419 ppm +/− 5% |
| Potassium | 398 ppm +/− 5% |
| Bicarbonate | 146 ppm +/− 5% |
| Bromide | 68 ppm +/− 5%. |

2. A method according to claim 1 wherein the concentration of magnesium is between about 20,000 and 50,000 PPM.

3. A method according to claim 2 wherein the concentration of magnesium is about 44,000 PPM.

4. A method according to claim 1 wherein the added magnesium salt is present in at least one of magnesium oxide, magnesium carbonate, magnesium chloride, magnesium sulfate, magnesite and dolomite.

5. The method according to claim 4 wherein the added magnesium salt is at least one of magnesium chloride and magnesium sulfate.

6. An aqueous composition for the treatment of acne comprising:

| | |
|---|---|
| Chloride | 19087 ppm +/− 5% |
| Sodium | 11211 ppm +/− 5% |
| Sulfite | 2585 ppm +/− 5% |
| Magnesium | 8000 ppm to 100,000 ppm |
| Calcium | 419 ppm +/− 5% |
| Potassium | 398 ppm +/− 5% |
| Bicarbonate | 146 ppm +/− 5% |
| Bromide | 68 ppm +/− 5%. |
